(19) United States
(12) Patent Application Publication
(10) Pub. No.: US 2007/0163327 A1
(43) Pub. Date: Jul. 19, 2007

(54) Detection of trace amounts of airborne or deposited low-molecular weight compounds

(75) Inventor: Per Mansson, Sollentuna (SE)

Correspondence Address: BACON & THOMAS, PLLC, 625 SLATERS LANE, FOURTH FLOOR, ALEXANDRIA, VA, 22314, US

(73) Assignee: Biosensor Applications Swedeen AB (publ, Sundbyberg (SE)

(21) Appl. No.: 10/557,598

(22) PCT Filed: May 18, 2004
(86) PCT No.: PCT/SE04/00767

§ 371 Date: Dec. 11, 2006

Related U.S. Application Data
(60) Provisional application No. 60/472,424, filed on May 22, 2003.

(30) Foreign Application Priority Data
May 22, 2003 (SE) 0301519-5

(51) Int. Cl. G01N 1/40; G01N 1/22; G01N 33/15; B03C 3/38

(52) U.S. Cl. 73/31.02; 73/31.07; 73/104; 96/26; 96/95

(57) ABSTRACT

A system and a method for detection of trace amounts of airborne or deposited low-molecular weight compound(s), such as narcotics or explosives, comprising an electrically charged collector surface connected to an ionizer ionizing airborne particles with an electrical charge opposite to the charge of the collector surface, optional air-stirring means, extraction means for extraction of collected material from the collector surface, and equipment for chemical and/or biochemical detection of said compound(s) from said extracted collected material, is disclosed. Further, a modified ionizer/collector device wherein the collector is extended from the body of the device and is equipped with a preferably detachable, electrically chargeable collecting surface and wherein optionally the air-ionizing part of the device is also extended from the body of the ionizer/collector device, either together with or apart from, the extended collector arm, is described. The device may be battery-driven and portable, and suitable for use by the customs, airport security and police for the detection of e.g. narcotics or explosives or in mine detection.

The present invention is concerned with the detection of trace amounts of airborne or deposited low-molecular weight compounds.

### BACKGROUND

Air ionization apparatuses have been used for production of ions in air by emitting electrons from a negatively charged metal surface. Negatively charged particles have then been collected on a positively charged surface of the apparatuses and the collected particles, such as bacteria, mould, fungal matter and in particular smoke particles, have been discarded e.g. as disclosed in WO 97/50160.

In WO 02/35209 is disclosed a device for collecting charged particles, e.g. mould spores, bacteria, viruses, dust, pollen, smoke, plant parts, fungus spores, allergens, and particles from animals with the aid of an ionizer for subsequent analysis in a particle analyzer established for this purpose, e.g. a scanning electron microscope.

### DESCRIPTION OF THE INVENTION

The present invention is directed to a system and a method of detecting trace amounts of airborne or deposited low-molecular weight compounds.

The invention is, in one aspect, directed to a system for detection of trace amounts of airborne or deposited low-molecular weight compound(s), comprising an electrically charged collector surface connected to an ionizer ionizing airborne particles with an electrical charge opposite to the charge of the collector surface, optional air-stirring means, extraction means for extraction of collected material from the collector surface, and equipment for chemical and/or biochemical detection of said compound(s) from said extracted collected material.

In an embodiment of the system the electrically charged collector surface is positively charged, the air-stirring means is selected from mechanical taping on close-by surfaces, pressurized air and a fan, the extraction means is selected from evaporation and condensation means and/or rinsing means for rinsing with a liquid to form a liquid sample for analysis, and said equipment for chemical and/or biochemical detection of said compound(s) from said liquid sample is selected from equipment for mass spectroscopy (MS), desorption MS, gas chromatography (GC), GC-MS, liquid chromatography (LC), UV, immunochemistry, piezoelectric microbalances with biocells, and surface plasmon resonance (SPR).

In an other embodiment of the system the low-molecular weight compound(s) is (are) selected from the group consisting of narcotics, such as cocaine, heroin, amphetamine, methamphetamine, tetrahydrocannabinols (THC), and methylenedioxy-N-methamphetamine (Ecstacy), and explosives, such as trinitrotoluene (TNT), dinitrotoluene (DNT), hexahydro-1,3,5-trinitro -1,3,5-triazine (RDX), octahydro-1,3,5,7-tetranitro-1,3,5,7-tetrazine (HMX), pentaerythritol tetranitrate (PETN), and nitroglycerine (NG).

Another aspect of the invention is directed to a method for detecting trace amounts of airborne or deposited low-molecular weight compound(s), comprising collecting particles from air with an electrically charged collector surface connected to an ionizer ionizing airborne particles with an electrical charge opposite to the charge of the collector surface, optionally stirring air in the vicinity of possibly deposited low-molecular weight compound(s), extracting the collected material from the collector surface, and analyzing the extracted material by chemical and/or biochemical methods.

In an embodiment of the method the electrically charged collector surface is positively charged, the stirring of air is effected by mechanical taping on close-by surfaces, with a stream of pressurized air or with a fan, the extraction of the collected material from the collector surface is effected by evaporation and condensation and/or rinsing with a liquid to form a liquid sample for analysis, and chemical and/or biochemical detection of said compound(s) from said liquid sample is selected from mass spectroscopy (MS), desorption MS, gas chromatography (GC), GC-MS, liquid chromatography (LC), UV, immunochemistry, piezoelectric microbalances with biocells, and surface plasmon resonance (SPR).

In another embodiment of the method compound(s) is (are) -selected from the group consisting of narcotics, such as cocaine, heroin, amphetamine, methamphetamine, tetrahydrocannabinols (THC), and methylenedioxy-N-methamphetamine (Ecstacy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

The term “low-molecular weight” in connection with the word “compounds” is intended to indicate that the compounds in question have molecular weights of 1000 or less.

Air in the vicinity of a suspected or investigated area or source of (a) deposited low-molecular weight compound(s) of interest is ionized with an ionizer, and charged airborne particles are collected on a collecting surface having an opposite charge to the charged particles.

The collecting surface is then either heated to evaporate the compound(s) of interest and subsequently condensed and/or extracted with a liquid to form a liquid sample for analysis.

In a preferred embodiment the air from which particles are to be collected, is made turbulent by some kind of stirring, such as mechanical taping on close-by surfaces or with pressurized air or with a fan. This stirring will promote movement of deposited particles into the air.

The present invention is also directed to a modified ionizer/collector device wherein the collector is extended from the body of the device and is equipped with a preferably detachable, electrically chargeable collecting surface. Electricity is supplied to ionizer/collector device e.g. with batteries. A battery-driven ionizer/collector device is portable and facilitates its use in all kinds of applications.

Thus, yet another aspect of the invention is directed to an ionizer device comprising an electrically charged collector surface connected to an ionizer unit ionizing airborne particles with an electrical charge opposite to the charge of the collector surface, wherein the collector surface is extended from the body of the ionizer by an insulated electrically conducting flexible arm.

In an embodiment of the ionizer device the ionizer unit ionizing airborne particles is extended from the body of the ionizer by an insulated electrically conducting flexible arm.

In another embodiment the flexible arm(s) is (are) detachable from the ionizer.

In still another embodiment the collector surface is detachable from the electrically conducting flexible arm.

Thus, the air-ionizing part of the device is also extended from the body of the ionizer/collector device, either together with or apart from, the extended collector arm.

The modified ionizer/collector device of the invention is particularly useful when sampling is made from closed or nearly closed compartments and the extension(s) is (are) stuck into said compartment(s) without having to make room for the whole ionizer/collector device. Non-limiting examples of such compartments are cars, cargo and containers.

The invention is particularly concerned with the detection of narcotics, such as cocaine, heroin, amphetamine, methamphetamine, tetrahydrocannabinols (THC), and methylenedioxy-N-methamphetamine (Ecstacy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

The invention may thus be used e.g. by the customs, airport security and police for the detection of narcotics or explosives, or in mine detection.

The liquid samples containing the collected particles, such as dust particles and/or droplets of air humidity, carrying, e.g. by adhesion, low-molecular weight compounds of interest, can be subjected to qualitative and/or quantitative determination of the presence of the compound or compounds of interest by chemical and/or biochemical methods, such as mass spectroscopy (MS), desorption MS, gas chromatography (GC), G G-MS, liquid chromatography (LC), UV, immunochemistry, piezoelectric microbalances with blocells, surface plasmon resonance (SPR) etc. The collected particles are probably homogenous explosives or homogenous narcotics particles.

In a preferred embodiment the compound or compounds of interest are extracted and dissolved in the rinsing liquid prior to analysis.

The teachings of all cited patent applications mentioned in this specification are incorporated herein by reference.

The invention is further illustrated by the following experiments and accompanied drawings comprising FIGS. 1-9.

### SHORT DESCRIPTION OF THE DRAWINGS

FIG. 1. Ionizer with the collector metal tip.

FIG. 2. Ionizer of FIG. 1 placed in a car

FIG. 3. A typical response from a biocell of a sample from a ionizer experiment.

FIG. 4. “The wooden box (Box I ; 120 liters) used in the sampling experiments”

FIG. 5. Results from sampling using a conventional BiosensD sampling unit.

FIG. 6. Results from sampling using a gold-coated filter connected to the positive collector of the ionizer.

FIG. 7. Results from sampling using the metal tip.

FIG. 8. Results from sampling using the metal tip after 1 week. Two consecutive samplings from the closed volume (Boxes) were performed as indicated in the figure. Samplings from the environment (slightly contaminated?) were also performed.

FIG. 9. Results from sampling of cocaine using the metal tip after 24 h in a 100 liter-box. (10 mg cocaine were blown out into the closed 100 liter-box)

### SAMPLING OF TRACES OF COCAINE FROM A CAR WITH THE AID OF AN IONIZER/COLLECTOR DEVICE

### Background

The objective of this test is to determine if it is possible to collect traces of drugs by using an ionizer together with a collector, a device essentially disclosed in WO 02/35209. A car was contaminated with small amounts of cocaine, and traces collected from air were subjected to chemical/biochemical analysis after 1-10 days.

### EXPERIMENTAL

Material:

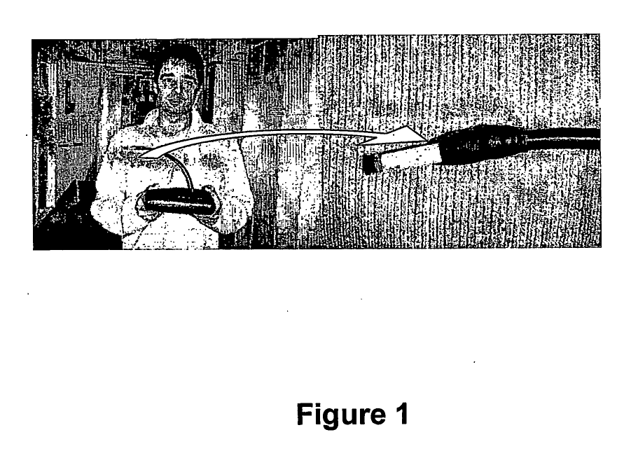

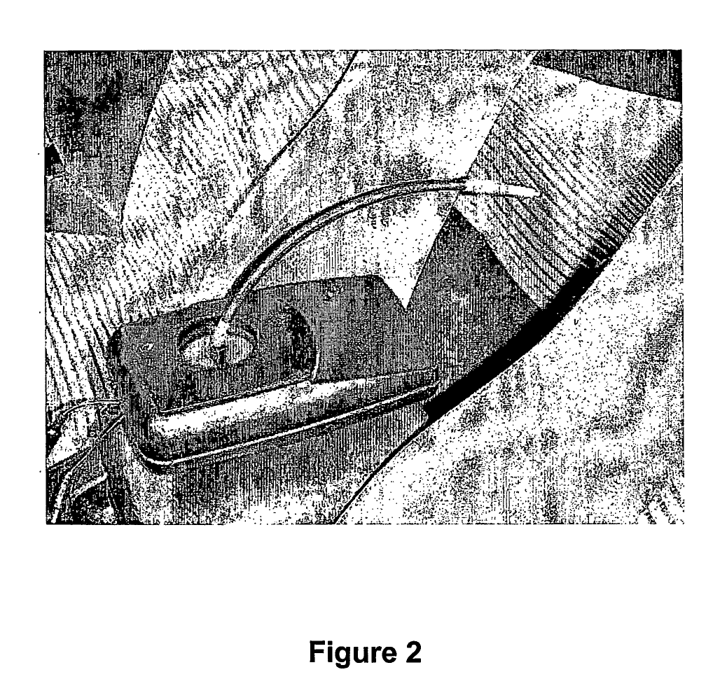

A car (Toyota Camry) was polluted with 2 mg cocaine by smearing it out onto the back seat, subsequently detected using three different sampling methods and chemical/biochemical analysis.
1. BiosensD collection unit equipped with a filter for air-sampling (sampling time: 60 sec) [in principal a vacuum cleaner with a filter disclosed in our co-pending patent application PCT/SE03/00319]
2. Ionization/collection unit (disclosed in WO 02/35209 but modified by −extension of the positively charged collector means, See FIG. 1) (sampling time 15 60 sec). The ionization/collection unit was placed in the middle of the car between the front seats for sampling of particles from air (See FIG. 2).
3. Wiping of surfaces in the car with white filters. Windows and plane “plastic” surfaces were wiped for 10 sec.
   Sampling and Analysis

We performed sampling of the car after 1 hour and after one week using the three sampling techniques described above. The filters and the material collected by using the ionizer/collector were analysed using a standard BIOSENS detection instrument, i.e. a piezoelectric microbalance with flow-through cells activated with antibodies specific for target compounds, described in detail in our co-pending Swedish patent application 0201877-8. The filters were analysed by subjecting them to thermal desorption and condensation on a cold finger essentially as disclosed in our co-pending patent application PCT/SE02/02098. The material collected onto the metal tip of the positively charged collector extension arm of the ionizer was extracted by detaching the metal tip and placing it directly into the extraction device (bath) in the same way as the cold fingers are extracted in the BIOSENS instrument.

Results and Discussions

A significant amount of cocaine was detected after 1 week after the deposition of the cocaine powder. It is important to note that it is necessary to whip the textile on the back seat in the car before the sampling. Only a minor response was detected when the sampling was performed without the whip-procedure.

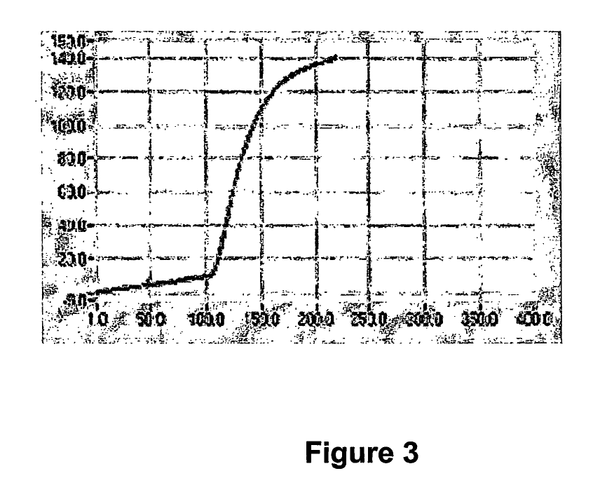

The results from the sampling/analysis experiments are tabulated in Table 1 and presented in FIG. 3.

TABLE 1
Responses from sampling and analysis of the cocaine polluted car

| Time after | | Sampling | Sampling | Response |
|---|---|---|---|---|
| deposition | | technique | time | (Hz) |
| 60 | min | filter | 1 min | 75 |
| 60 | min | ionizer | 1 min | >100 |
| 20 | h | filter | 30 sec | 35 |
| 20 | h | ionizer | 30 sec | 50 |
| 100 | h | ionizer | 30 sec | >100 |
| 1 | week | (1) filter | 30 sec | 30-40 |
| 1 | week | (2) ionizer | | 30-40 |
| 1 | week | “Wiping” | 10 sec | 10-40 |
| | | (two | | |
| | | filters) | | |

Sampling of Cocaine and Ecstacy with the Aid of an Ionizer/Collector Device

### BACKGROUND

The objective of this test is to test a new technique to collect drugs by using the same ionizer device together with a collector disclosed above.

### EXPERIMENTAL

Material:

A filter was coated with gold and placed in firm contact with the positive collector on the ionizer via a metal tip made (see FIG. 1).

A number of sampling experiments were performed using a metal tip that was connected to the positive electrode on the ionizer (collecting area 0.5 cm²). See FIG. 1

Sampling Experiments

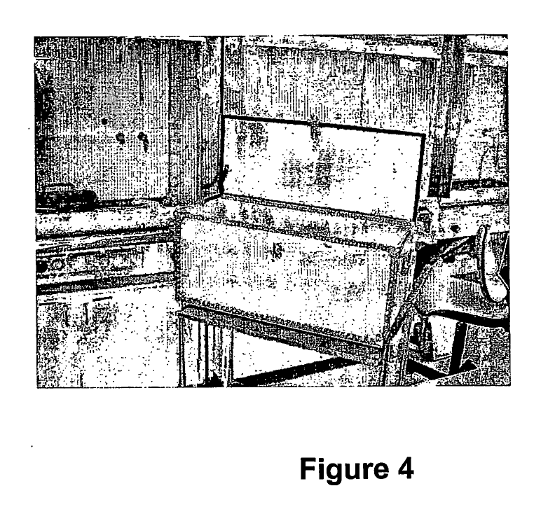

Two wooden boxes (Box I and Box II see FIG. 4) (volume 120 and 100 liters, respectively) were used for a number of experiments. The boxes were loaded with 10 mg of cocaine or ecstacy (10 mg) by blowing it out into the box volume.

60 minutes after the loading a sampling was performed using the BiosensD sampling unit (Filter) (100 liters/sec, 3 minutes). After an additional 60 min, a second sample was collected by using the ionizer equipped with a gold-coated filter (4 min). The third and fourth samplings were performed by using a metal tip connected to the collector of the ionizer device (4 min. sampling time).

The filters from the two first samplings (filter-sampling) from Box I above were desorbed on a BiosensD-instrument (desorption parameters were optimised for ecstasy, 210° C., 15 sec) and extracted by using 100 microliters of PBS, and subsequently analysed by loop injection into activated ecstasy cells and activated cocaine cells.

In the sampling experiments using the ionizer, the metal tips were extracted directly with 100 microliters of PBS without desorption prior to the analysis by loop-injecting the samples into the flow of fluid entering the biocells, which were activated with MAB (monoclonal antibodies specific for cocaine and ecstacy, respectively) before each experiment.

Experiments in Wooden Boxes I and II (See FIG. 3).

The boxes were loaded as described above with 10 ng cocaine or 10 mg ecstasy.

After 24h the cocaine in the boxes was collected by using the ionizer/metal tip collector. Both the ionizer part and the collector metal tip were moving about 2 cm from the surfaces in the box for 15 sec to 2 minutes or placed stationary in the box for 1 minute. The metal tip was extracted with about 100 microliters of buffer and introduced to the activated biocell.

Sampling from Textiles

An office chair was contaminated with 2 mg of cocaine by dissolving the cocaine in methanol and subsequently impregnating an area of 15 cm×15 cm with the methanol solution. All sampling experiments were performed after 3h by using the ionizer/metal tip collector and conventional filter. The contaminated area was “scratched” and whipped with my fingers a few seconds prior to the sampling procedure. Sampling times were 15 sec to 3 minutes.

Results and Discussions

Box I.

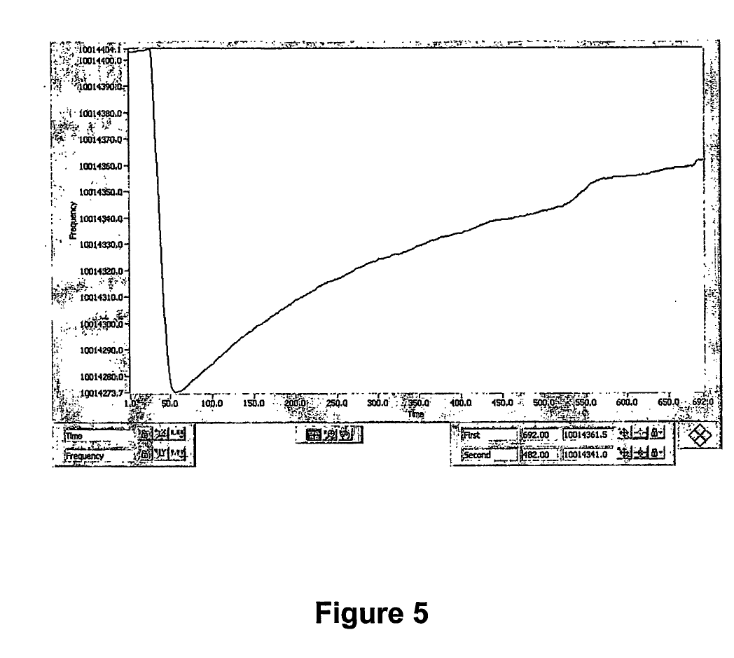

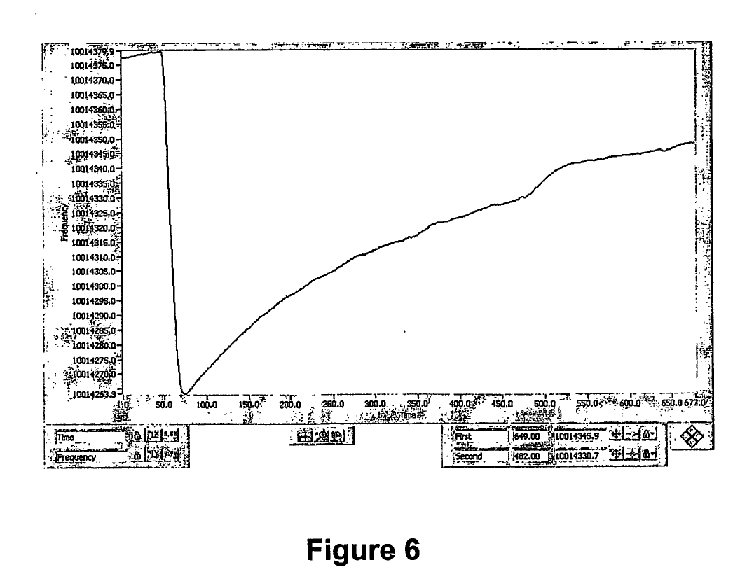

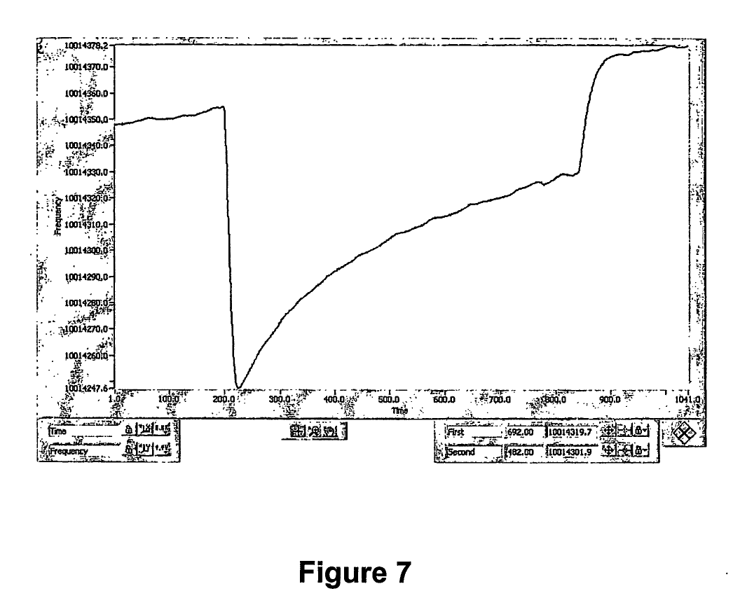

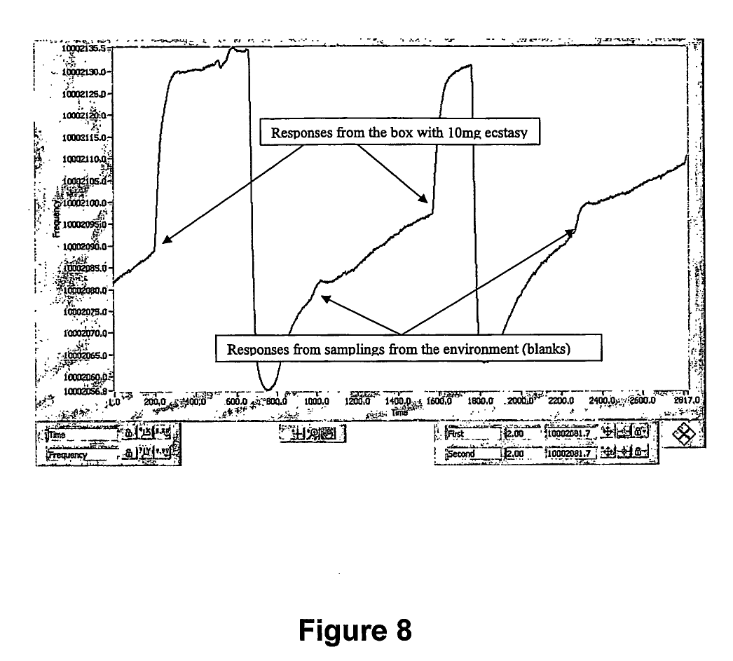

The box was loaded with ecstasy (10 mg). We found ecstasy in all sampling experiments. The amount of ecstasy from the sampling using ionizer equipped with a metal tip was superior to the two other samplings using a conventional filter (BiosensD sampler) and ionizer/gold-coated filter. Sampling using the conventional BiosensD-device and the ionizer with gold filter showed about the same response (7 Hz). The response from the metal tip showed about 30-40 Hz both after one hour and one week. Blank analysis of clean metal tips (blanks) outside the boxes did not show any responses. The results can be seen in FIGS. 5, 6, 7 and 8, respectively.

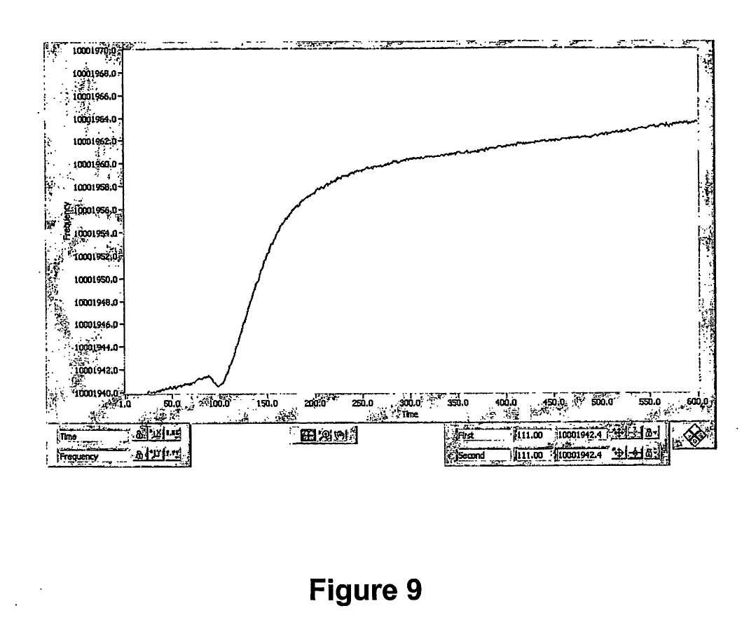

A number of separate experiments using 10 mg cocaine in Box II were performed 24h after the loading procedure. The collector tip on the ionizer was extracted and analysed in a BiosensD instrument equipped with Biocells sensitive for cocaine, heroin, ecstasy and amphetamine, respectively. The cocaine cell always showed very high responses (30-150 Hz) indicating a very large amount of cocaine on the tip. No responses were detected from biocells, that were activated for heroin, ecstasy or amphetamines The results from the cocaine cell can be seen in FIG. 9.

1. System for detection of trace amounts of airborne or deposited low-molecular weight compound(s), comprising an electrically charged collector surface connected to an ionizer ionizing airborne particles with an electrical charge opposite to the charge of the collector surface, optional air-stirring means, extraction means for extraction of collected material from the collector surface, and equipment for chemical and/or biochemical detection of said compound(s) from said extracted collected material.

2. System according to claim 1, wherein the electrically charged collector surface is positively charged, the air-stirring means is selected from mechanical taping on close-by surfaces, pressurized air and a fan, the extraction means is selected from evaporation and condensation means and/or rinsing means for rinsing with a liquid to form a liquid sample for analysis, and said equipment for chemical and/or biochemical detection of said compound(s) from said liquid sample is selected from equipment for mass spectroscopy (MS), desorption MS, gas chromatography (GC), GC-MS, liquid chromatography (LC), UV, immunochemistry, piezoelectric microbalances with biocells, and surface plasmon resonance (SPR).

3. System according to claim 1, wherein the low-molecular weight compound(s) is (are) selected from the group consisting of narcotics, such as cocaine, heroin, amphetamine, methamphetamine, tetrahydrocannabinols (THC), and methylenedioxy-N-methamphetamine (Ecstasy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

4. Method for detecting trace amounts of airborne or deposited low-molecular weight compound(s), comprising collecting particles from air with an electrically charged collector surface connected to an ionizer ionizing airborne particles with an electrical charge opposite to the charge of the collector surface, optionally stirring air in the vicinity of possibly deposited low-molecular weight compound(s), extracting the collected material from the collector surface, and analyzing the extracted material by chemical and/or biochemical methods.

5. Method according to claim 4, wherein the electrically charged collector surface is positively charged, the stirring of air is effected by mechanical taping on close-by surfaces, with a stream of pressurized air or with a fan, the extraction of the collected material from the collector surface is effected by evaporation and condensation and/or rinsing with a liquid to form a liquid sample for analysis, and chemical and/or biochemical detection of said compound(s) from said liquid sample is selected from mass spectroscopy (MS), desorption MS, gas chromatography (GC), GC-MS, liquid chromatography (LC), UV, immunochemistry, piezoelectric microbalances with biocells, and surface plasmon resonance (SPR).

6. Method according to claim 4, wherein the low-molecular weight compound(s) is (are) selected from the group consisting of narcotics, such as cocaine, heroin, amphetamine, methamphetamine, tetrahydrocannabinols (THC), and methylenedioxy-N-methamphetamine (Ecstasy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

7. Ionizer device comprising an electrically charged collector surface connected to an ionizer unit ionizing airborne particles with an electrical charge opposite to the charge of the collector surface, wherein the collector surface is extended from the body of the ionizer by an insulated electrically conducting flexible arm.

8. Ionizer device according to claim 7, wherein the ionizer unit ionizing airborne particles is extended from the body of the ionizer by an insulated electrically conducting flexible arm.

9. Ionizer device according to claim 7, wherein the flexible arm(s) is (are) detachable from the ionizer.

10. Ionizer device according to claim 7, wherein the collector surface is detachable from the electrically conducting flexible arm.

11. System according to claim 2, wherein the low-molecular weight compound(s) is (are) selected from the group consisting of narcotics, such as cocaine, heroin, amphetamine, methamphetamine, tetrahydrocannabinols (THC), and methylenedioxy-N-methamphetamine (Ecstasy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

12. Method according to claim 5, wherein the low-molecular weight compound(s) is (are) selected from the group consisting of narcotics, such as cocaine, heroin, amphetamine, methamphetamine, tetrahydrocannabinols (THC), and methylenedioxy-N-methamphetamine (Ecstasy), and explosives, such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

13. Ionizer device according to claim 8, wherein the flexible arm(s) is (are) detachable from the ionizer.

14. Ionizer device according to claim 8, wherein the collector surface is detachable from the electrically conducting flexible arm.

15. Ionizer device according to claim 9, wherein the collector surface is detachable from the electrically conducting flexible arm.
